(19) United States
(12) Patent Application Publication
(10) Pub. No.: US 2026/0196291 A1
(43) Pub. Date: Jul. 9, 2026

(54) TECHNOLOGIES FOR TAXONOMY-AWARE EMBEDDING AND PSEUDO-IMAGE REPRESENTATION FOR ZERO-SHOT LEARNING GENOME CLASSIFICATION

(71) Applicants: AUBURN UNIVERSITY, Auburn, AL (US); THE BOARD OF REGENTS FOR THE OKLAHOMA AGRICULTURAL AND MECHANICAL COLLEGES, Oklahoma City, OK (US)

(72) Inventors: Sathyanarayanan N. Aakur, Auburn, AL (US); Vishalini Laguduva Ramnath, Auburn, AL (US); Akhilesh Ramachandran, Stillwater, OK (US)

(21) Appl. No.: 19/444,395

(22) Filed: Jan. 9, 2026

Related U.S. Application Data
(60) Provisional application No. 63/743,415, filed on Jan. 9, 2025.

(51) Int. Cl. G16B 10/00; G16B 30/00; G16B 40/30; G16B 45/00

(52) U.S. Cl. CPC G16B 10/00; G16B 30/00; G16B 40/30; G16B 45/00

(57) ABSTRACT

Technologies for taxonomy-aware embedding and pseudo-image representation for zero-shot learning genome classification includes a computing device that learns an embedding space based on a taxonomic graph using a feature learning algorithm. The taxonomic graph includes nodes representing levels in a hierarchical taxonomy, such as a taxonomy of organisms reflecting phylogenetic relationships between the organisms. The computing device determines a pseudo-image representation of a whole genome of an organism based on the co-occurrence frequency of pairs of k-mers selected from the sequence reads indicative of the whole genome of the organism. The computing device trains a classification model to map the pseudo-image representation to the embedding space. The computing device uses the trained classification model to map a query genome to a vector representation in the embedding space. Other embodiments are described and claimed.

### CROSS-REFERENCE TO RELATED APPLICATIONS

The present application claims priority under 35 U.S.C. § 119 (e) to U.S. Provisional Application Ser. No. 63/743,415, filed Jan. 9, 2025, the entire disclosure of which is hereby incorporated by reference.

### BACKGROUND

In microbiology, whole and partial genome sequencing is frequently used to study the origins, pathogenicity, and phylogenetic relationships of bacterial and viral organisms. However, the vast number of potential species poses significant challenges in developing a scalable and efficient whole genome classification tool. Traditional bioinformatics methods typically involve matching nucleotide sequences to a predetermined search space and calculating the statistical significance of the matches, which can be time-consuming and resource-intensive. Machine learning approaches generally require thousands of examples per class to achieve reasonable performance, which is impractical given the extremely large number of potential classes (e.g., the estimated number of bacterial species ranging from 10⁷ to 10⁹) and the sparseness of labeled data. Additionally, the long-tail distribution of labeled data means that not all species are equally represented, making it challenging to capture the entire spectrum of species.

### SUMMARY

According to one aspect of the disclosure, a computing device for representing a taxonomy in a vector space includes an embedding model trainer and an embedding mapper. The embedding model trainer is configured to construct a taxonomic graph and to learn an embedding space based on the taxonomic graph with a feature learning algorithm. The taxonomic graph includes a plurality of nodes indicative of levels in a hierarchical taxonomy and a plurality of edges that connect nodes. The embedding mapper is configured to generate a first vector representation of a first species in response to learning of the embedding space. The first vector representation includes a plurality of features in the embedding space.

In one embodiment, to learn the embedding space includes to learn a mapping from a node in the taxonomic graph to a feature representation in the embedding space. To generate the first vector representation includes to apply the mapping to a first node in the taxonomic graph indicative of the first species.

In one embodiment, the hierarchical taxonomy includes a taxonomy of organisms reflecting phylogenetic relationships between the organisms. In one embodiment, to construct the taxonomic graph includes to recursively construct the taxonomic graph based on a predetermined plurality of organisms.

According to another aspect, a computing device for representing a genomic sequence includes a sequence analyzer and a pseuodo-image factory. The sequence analyzer is configured to determine a plurality of k-mers from a plurality of sequence reads and to determine a relative co-occurrence between each pair of k-mers of the plurality of k-mers. The plurality of sequence reads is indicative of a first whole genome of a first organism. Each k-mer includes an atomic sub-sequence of nucleotide bases having a predetermined length. The pseuodo-image factory is configured to construct a first pseudo-image as a function of the relative co-occurrence between each pair of k-mers. Each pixel of the first pseudo-image has a value indicative of the relative co-occurrence for an associated pair of k-mers.

In one embodiment, to construct the first pseudo-image comprises includes to construct a plurality of intermediate pseudo-images, wherein each intermediate pseudo-image is generated for a corresponding sequence read, and to sum pixel values of the plurality of intermediate pseudo-images to generate the first pseudo-image. In one embodiment, to construct the first pseudo-image includes to concatenate the plurality of sequence reads to generate a concatenated sequence read, and to construct the first pseudo-image with the concatenated sequence read. In one embodiment, to construct the first pseudo-image includes to construct a plurality of intermediate pseudo-images, wherein each intermediate pseudo-image of the plurality of intermediate pseudo-images is generated for a corresponding sequence read, to construct a second plurality of intermediate pseudo-images, wherein each intermediate pseudo-image of the second plurality is generated for a reverse complement of a corresponding sequence read, and to sum pixel values of the plurality of intermediate pseudo-images and the second plurality of intermediate pseudo-images to generate the first pseudo-image.

In one embodiment, to determine the plurality of k-mers includes to select one or more k-mers from each sequence read using a sliding window of the predetermined length and a predetermined stride.

In one embodiment, to determine the relative co-occurrence between each pair of k-mers includes to iteratively update the relative co-occurrence for each pair of k-mers with an update function. In one embodiment, in the update function, an initial relative co-occurrence value for each pair of k-mers is set to zero, the relative co-occurrence value for each pair of k-mers is set to one when the pair of k-mers is first observed, the relative co-occurrence value for each pair of k-mers is increased when the pair of k-mers is subsequently observed, and output of the update function is bound by a weighted update function.

In one embodiment, each of the sequence reads comprises a DNA sequence. In one embodiment, each of the sequence reads comprises a 16S RNA sequence, a 23S RNA sequence, or a metagenome sequence.

According to another aspect, a computing device for classification of organisms by species includes an embedding model trainer, a classifier model trainer, a pseudo-image factory, and a classifier. The embedding model trainer is configured to learn an embedding space based on a taxonomic graph with a feature learning algorithm. The taxonomic graph includes a plurality of nodes indicative of levels in a hierarchical taxonomy and a plurality of edges that connect nodes. The classifier model trainer is configured to train a classifier model to map a pseudo-image indicative of a whole genome of an organism to a corresponding vector representation in the embedding space. The pseudo-image factory is configured to generate a first pseudo-image indicative of a first whole genome of a first organism. The classifier is configured to determine a first vector representation of a first species associated with the first whole genome by input of the first pseudo-image to the classifier model in response to training of the classifier model. In one embodiment, the classifier model comprises a convolutional neural network.

In one embodiment, the computing device further includes a classification analyzer configured to search for a second node in the hierarchical taxonomy based on a distance in the embedding space between the first vector representation and a second vector representation of the second node. In one embodiment, the second node comprises a second species.

According to another aspect, a method for representing a taxonomy in a vector space includes constructing, by a computing device, a taxonomic graph, wherein the taxonomic graph comprises a plurality of nodes indicative of levels in a hierarchical taxonomy and a plurality of edges that connect nodes; learning, by the computing device, an embedding space based on the taxonomic graph with a feature learning algorithm; and generating, by the computing device, a first vector representation of a first species in response to learning the embedding space, wherein the first vector representation comprises a plurality of features in the embedding space.

In one embodiment, learning the embedding space includes learning a mapping from a node in the taxonomic graph to a feature representation in the embedding space, and generating the first vector representation includes applying the mapping to a first node in the taxonomic graph indicative of the first species.

In one embodiment, the hierarchical taxonomy comprises a taxonomy of organisms reflecting phylogenetic relationships between the organisms. In one embodiment, constructing the taxonomic graph includes recursively constructing the taxonomic graph based on a predetermined plurality of organisms

According to another aspect, a method for representing a genomic sequence includes determining, by a computing device, a plurality of k-mers from a plurality of sequence reads, wherein the plurality of sequence reads is indicative of a first whole genome of a first organism, and wherein each k-mer comprises an atomic sub-sequence of nucleotide bases having a predetermined length; determining, by the computing device, a relative co-occurrence between each pair of k-mers of the plurality of k-mers; and constructing, by the computing device, a first pseudo-image as a function of the relative co-occurrence between each pair of k-mers, wherein each pixel of the first pseudo-image has a value indicative of the relative co-occurrence for an associated pair of k-mers.

In one embodiment, constructing the first pseudo-image includes constructing a plurality of intermediate pseudo-images, wherein each intermediate pseudo-image is generated for a corresponding sequence read; and summing pixel values of the plurality of intermediate pseudo-images to generate the first pseudo-image. In one embodiment, constructing the first pseudo-image includes concatenating the plurality of sequence reads to generate a concatenated sequence read; and constructing the first pseudo-image with the concatenated sequence read. In one embodiment, constructing the first pseudo-image includes constructing a plurality of intermediate pseudo-images, wherein each intermediate pseudo-image of the plurality of intermediate pseudo-images is generated for a corresponding sequence read; constructing a second plurality of intermediate pseudo-images, wherein each intermediate pseudo-image of the second plurality is generated for a reverse complement of a corresponding sequence read; and summing pixel values of the plurality of intermediate pseudo-images and the second plurality of intermediate pseudo-images to generate the first pseudo-image.

In one embodiment, determining the plurality of k-mers includes selecting one or more k-mers from each sequence read using a sliding window of the predetermined length and a predetermined stride.

In one embodiment, determining the relative co-occurrence between each pair of k-mers includes iteratively updating the relative co-occurrence for each pair of k-mers using an update function. In one embodiment, in the update function, an initial relative co-occurrence value for each pair of k-mers is set to zero, the relative co-occurrence value for each pair of k-mers is set to one when the pair of k-mers is first observed, the relative co-occurrence value for each pair of k-mers is increased when the pair of k-mers is subsequently observed, and output of the update function is bound by a weighted update function.

In one embodiment, each of the sequence reads comprises a DNA sequence. In one embodiment, each of the sequence reads comprises a 16S RNA sequence, a 23S RNA sequence, or a metagenome sequence.

According to another aspect, a method for classifying organisms by species includes learning, by a computing device, an embedding space based on a taxonomic graph with a feature learning algorithm, wherein the taxonomic graph comprises a plurality of nodes indicative of levels in a hierarchical taxonomy and a plurality of edges that connect nodes; training, by the computing device, a classifier model to map a pseudo-image indicative of a whole genome of an organism to a corresponding vector representation in the embedding space; generating, by the computing device, a first pseudo-image indicative of a first whole genome of a first organism; and determining, by the computing device, a first vector representation of a first species associated with the first whole genome by inputting the first pseudo-image to the classifier model in response to training the classifier model. In one embodiment, the classifier model comprises a convolutional neural network.

In one embodiment, the method further includes searching, by the computing device, for a second node in the hierarchical taxonomy based on a distance in the embedding space between the first vector representation and a second vector representation of the second node. In one embodiment, the second node comprises a second species.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for taxonomy-aware embedding and pseudo-image representation for zero-shot learning genome classification;

FIG. 2 is a simplified block diagram of at least one embodiment of an environment that may be established by a computing device of FIG. 1;

FIG. 3 is a simplified flow diagram of at least one embodiment of a method for embedding and classification model training that may be executed by the computing device of FIGS. 1 and 2;

FIG. 4 is a simplified flow diagram of at least one embodiment of a method for learning a taxonomy-aware embedding space that may be executed by the computing device of FIGS. 1 and 2;

FIG. 5 is a simplified flow diagram of at least one embodiment of a method for constructing a pseudo-image representation of a whole organism genome that may be executed by the computing device of FIGS. 1 and 2;

FIG. 6 is a simplified flow diagram of at least one embodiment of an iterative update method that may be executed in connection with the method of FIG. 5;

FIG. 7 is a simplified flow diagram of at least one embodiment of a method for genome classification using pseudo-image representations and a taxonomy-aware embedding space that may be executed by the computing device of FIGS. 1 and 2;

FIG. 8 is a schematic diagram illustrating one potential embodiment of a hierarchical taxonomic graph that may be used by the computing device of FIGS. 1 and 2 for generating the taxonomy-aware embedding;

FIG. 9 is a schematic diagram illustrating potential embodiments of pseudo-image representations that may be generated by the computing device of FIGS. 1 and 2; and

FIG. 10 is a pseudocode diagram illustrating one potential embodiment of a method for creating a pseudo-image representation of whole genome sequence reads.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

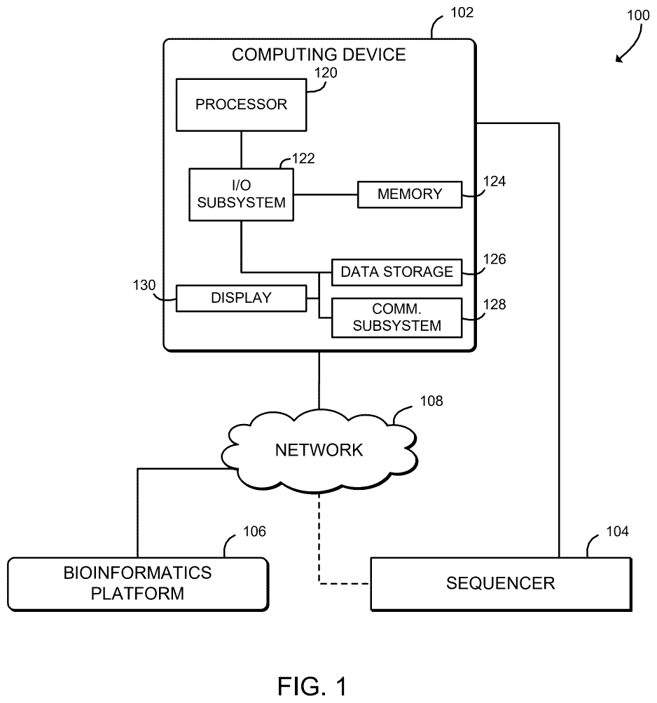

Referring now to FIG. 1, an illustrative system 100 for taxonomy-aware embedding and pseudo-image representation for zero-shot learning genome classification includes a computing device 102, which may be in communication with one or more sequencers 104 and/or bioinformatics platform 106 over a network 108. In use, as described further below, the computing device 102 generates a graph representation of taxonomic hierarchy and then learns an embedding space based on that taxonomic graph. This embedding space is taxonomy-aware, capturing the hierarchical and compositional relationships between species, genus, and so on. The computing device 102 further generates a pseudo-image representation of a whole genome of an organism. The computing device 102 uses the learned embedding space to generate embeddings for species in a sparse training set, and then trains a classification model to map pseudo-image representations to vector representations in the embedding space. The trained classification model can be used to map pseudo-image representations from test genomes to vector representations. The computing device 102 may analyze the determined vector representations in the embedding space, for example to determine similarity to other species, to determine genus or other taxonomic level, or to perform other analysis.

Accordingly, as described above, the system 100 provides a taxonomy-aware embedding space. This feature introduces a novel embedding space that captures compositional and phylogenetic relationships of species. Unlike traditional embeddings like word2vec, this embedding space is constructed using a hierarchical graph structure derived from species taxonomy, enabling more accurate and generalizable genome classification. This approach has not been explored in prior works such as Kraken, BLAST, or Centrifuge.

The system 100 further provides pseudo-imaging of genomes. The disclosed system 100 represents genome sequences as pseudo-images to capture inter- and intra-species variations at the nucleotide read level. This method leverages Gray Level Co-occurrence Matrices (GLCM) to create species-specific patterns in images, which is a novel application in genome analysis. Previous works like MG2V and Kraken do not use image-based representations for genome data. As described further below, the system 100 may extend the pseudo-imaging technique to provide complement-aware pseudo-imaging. This feature includes the reverse complement of genome sequences to capture the inherent symmetry in DNA structures. This enhancement improves classification accuracy and is not found in other genome analysis tools like MG-NET or Centrifuge.

The disclosed system 100 applies zero-shot learning to genome classification, allowing the system 100 to recognize species not seen during training. This approach addresses the challenge of large classification vocabularies with long-tail distributions, which is a significant improvement over traditional supervised learning methods used in BLAST and Kraken.

In an illustrative embodiment, the disclosed system 100 constructs a taxonomic graph iteratively from the kingdom level to the species level, capturing the phylogenetic relationships between organisms. This graph-based approach is novel compared to the brute-force matching algorithms used in traditional bioinformatics tools.

In an illustrative embodiment, the disclosed system 100 introduces a genome-to-taxonomy mapping function that projects genome-level pseudo-images into a taxonomy-aware embedding space using convolutional neural networks (CNNs). This method ensures generalizable zero-shot classification performance, which is not addressed by other frameworks like MG2V or MG-NET.

Additionally, the framework of the disclosed system 100 is designed to handle the scalability issues associated with the vast number of potential species, making it computationally efficient compared to traditional bioinformatics pipelines like BLAST, which are computationally intensive and time-consuming.

As described further below, in some embodiments, the disclosed system 100 may integrate supervised contrastive learning. The addition of a supervised contrastive learning loss may improve the discriminative features learned for each species. This approach enhances the generalization capabilities of the model, which is not considered in other genome classification methods.

This illustrative disclosed system 100 improves computational efficiency compared to previous bioinformatics system. For example, the disclosed system 100 significantly reduces the time taken for genome classification compared to traditional methods like BLAST. The use of pseudo-imaging and taxonomy-aware embeddings allows for faster and more efficient genome analysis.

Additionally, the disclosed system 100 includes the potential for integration with bioinformatics pipelines. The disclosed system 100 offers opportunities for integration with existing bioinformatics tools like BLAST, serving as an initial filter to reduce search space and overall latency in taxonomic profiling. This integration is not addressed by other genome classification frameworks.

In some embodiments, the disclosed system 100 extends the pseudo-imaging technique with hyperspectral pseudo-imaging techniques. In some embodiments, the system 100 may generate hyperspectral pseudo-images representing genome sequences. Hyperspectral pseudo-images may include multiple channels of pseudo-image data, analogous to spectral channels in conventional hyperspectral imagery. Each channel of the Hyperspectral pseudo-image may include, for example, pseudo-image representations generated using k-mers with different stride lengths or other variations of the pseudo-image representations.

Accordingly, the disclosed system 100 provides multiple advantages over conventional bioinformatics systems, including conventional whole genome classification techniques. For example, the disclosed system 100 improves scalability. The disclosed system 100's zero-shot learning approach allows it to handle a vast number of species, addressing the scalability issues of traditional bioinformatics tools like Kraken and BLAST, which are computationally expensive and time-consuming. As another example, the disclosed system 100 improves computational efficiency. The disclosed system 100 significantly reduces the time required for genome classification compared to BLAST. For example, in an experiment, an embodiment of the system 100 had an average execution time per query of 8 seconds, whereas BLAST takes around 25 minutes for the same task.

Further, the system 100 improves generalization to unseen species. The disclosed system 100's taxonomy-aware embedding space allows it to generalize to species not seen during training, unlike traditional machine learning models like MG2V and MG-NET, which are limited to the species seen during training.

The system 100 provides accuracy at multiple taxonomic levels. The disclosed system 100 achieves high accuracy at various taxonomic levels (e.g., species, genus, family, order), with top-1 accuracy for unseen species reaching 11.69% at the species level and 100% at the order level in an experiment.

The system 100 provides an enhanced genome representation. The disclosed system 100's pseudo-imaging technique captures phylogenetic variations at the nucleotide level, providing a richer and more discriminative representation compared to sequence-based models like MG2V.

The system 100 reduces the training data requirement as compared to existing systems. In an experiment, the disclosed system 100 achieved robust performance with only five labeled examples per species, making it suitable for scenarios with limited labeled data.

The system 100 provides improved genomic similarity prediction. In an experiment, the disclosed system 100's predictions show high genome similarity with actual species, with an embodiment incorporating complement-aware pseudo-imaging achieving over 87% genome similarity for top-1 predictions.

Additionally, the system 100 supports versatility in genome types. The disclosed system 100 can be extended to handle 16S and 23S rRNA gene sequencing and raw metagenome sequences, making it versatile for various genomic analysis tasks.

As described above, the system 100 may also be integrated with existing tools. For example, the disclosed system 100 could serve as a coarse filter to reduce the search space for tools like BLAST, improving overall latency and performance in taxonomic profiling.

Additionally, the system 100 provides robust feature learning. The disclosed system 100's use of convolutional neural networks (CNNs) and potential integration with advanced architectures like ResNet and vision transformers (ViT) provides a robust framework for learning discriminative features from genome data.

Referring again to FIG. 1, the computing device 102 may be embodied as any type of device capable of performing the functions described herein. For example, the computing device 102 may be embodied as, without limitation, a server, a rack-mounted server, a blade server, a workstation, a network appliance, a web appliance, a desktop computer, a laptop computer, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across the network 108 and operating in a public or private cloud. Accordingly, although the computing device 102 is illustrated in FIG. 1 as embodied as a single computing device, it should be appreciated that the computing device 102 may be embodied as multiple devices cooperating together to facilitate the functionality described below. As shown in FIG. 1, the illustrative computing device 102 includes a processor 120, an I/O subsystem 122, memory 124, a data storage device 126, and a communication subsystem 128. Of course, the computing device 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and/or software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the computing device 102 may be embodied as any communication circuit, device, or collection thereof, capable of enabling communications between the computing device 102, the mobile computing devices 104, the personal sensor devices 106, the autonomous sensor device 108, and/or other remote devices. The communication subsystem 128 may be configured to use any one or more communication technologies (e.g., wireless or wired communications) and associated protocols (e.g., Ethernet, Bluetooth®, Bluetooth Low Energy (BLE), Wi-Fi®, WiMAX, 3G LTE, 5G, etc.) to effect such communication.

As shown in FIG. 1, the computing device 102 may include a display 130. The display 130 may be embodied as any type of display capable of displaying digital images or other information, such as a liquid crystal display (LCD), a light emitting diode (LED), a plasma display, a cathode ray tube (CRT), or other type of display device. In some embodiments, the display 130 may be coupled to a touch screen to allow user interaction with the computing device 102.

The sequencer 104 may be embodied as any instrument or other machine capable of generating genome sequence reads for an organism, such as Illumina, Nanopore (MinION), Single Molecule, Real-Time (SMR) sequencing, Ion Torrent, sequencers, or other sequencer technologies. As described further below, the sequence reads generated by the sequencer 104 may represent nucleic acid sequence reads, bisulfate treated nucleic sequence reads, DNA sequence reads, RNA sequence reads, metagenomic sequence reads, or other genomic sequence reads. As shown, the computing device 102 may be coupled directly to the sequencer 104 and/or may be connected to the sequencer 104 via the network 108.

Similarly, the bioinformatics platform 106 may be embodied as any server, cloud server, virtual server, or other service capable of providing sequence reads for an organism. Accordingly, the bioinformatics platform 106 may include components and devices commonly found in an application server, a cloud server, a database server, or similar computing device, such as a processor, an I/O subsystem, a memory, a data storage device, a communication subsystem, and/or a display or other I/O devices. Those individual components of the bioinformatics platform 106 may be similar to the corresponding components of the computing device 102, the description of which is applicable to the corresponding components of the bioinformatics platform 106 and is not repeated herein so as not to obscure the present disclosure. Similarly, in some embodiments the bioinformatics platform 106 may be embodied as a virtual server, a cloud instance, or other component operating in a public or private cloud.

As discussed in more detail below, the computing device 102, the sequencer 104, and/or the bioinformatics platform may be configured to transmit and receive data with each other and/or other devices of the system 100 over the network 108. The network 108 may be embodied as any number of various wired and/or wireless networks. For example, the network 108 may be embodied as, or otherwise include, a wired or wireless local area network (LAN), a wired or wireless wide area network (WAN), a cellular network, and/or a publicly-accessible, global network such as the Internet. As such, the network 108 may include any number of additional devices, such as additional computers, routers, stations, and switches, to facilitate communications among the devices of the system 100.

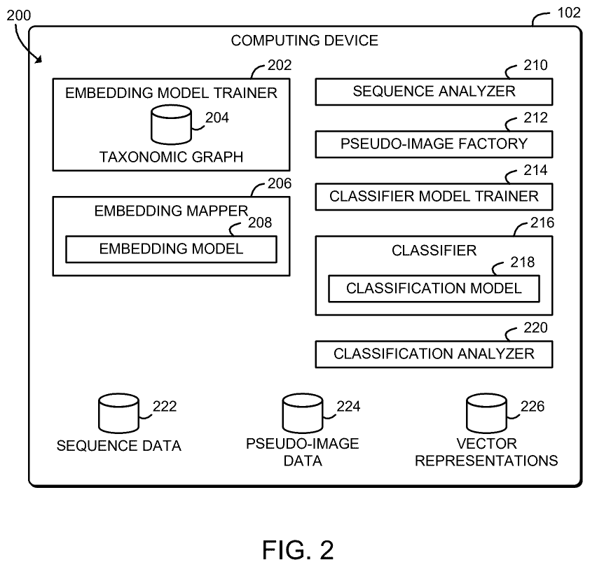

Referring now to FIG. 2, in the illustrative embodiment, the computing device 102 establishes an environment 200 during operation. The illustrative environment 200 includes an embedding model trainer 202, an embedding mapper 206, a sequence analyzer 210, a pseudo-image factory 212, a classifier model trainer 214, a classifier 216, and a classification analyzer 220. The various components of the environment 200 may be embodied as hardware, firmware, software, or a combination thereof. As such, in some embodiments, one or more of the components of the environment 200 may be embodied as circuitry or a collection of electrical devices (e.g., embedding model trainer circuitry 202, embedding mapper circuitry 206, sequence analyzer circuitry 210, pseudo-image factory circuitry 212, classifier model trainer circuitry 214, classifier circuitry 216, and/or classification analyzer circuitry 220). It should be appreciated that, in such embodiments, one or more of those components may form a portion of the processor 120, the I/O subsystem 122, and/or other components of the computing device 102.

The embedding model trainer 202 is configured to construct a taxonomic graph 204, which includes nodes indicative of levels in a hierarchical taxonomy and edges that connect the nodes. In an embodiment, the taxonomic graph 204 represents a taxonomy of organisms reflecting phylogenetic relationships between the organisms. In some embodiments, constructing the taxonomic graph 204 includes recursively construct the taxonomic graph 204 based on a predetermined selection of organisms. The embedding model trainer 202 is further configured to learn an embedding space based on the taxonomic graph with a feature learning algorithm. In some embodiments, learning the embedding space includes learning a mapping from a node in the taxonomic graph to a feature representation in the embedding space. This mapping from to node to feature representation may be performed by or otherwise configured in an embedding model 208.

The embedding mapper 206 is configured to generate a vector representation 226 of a species in response to learning the embedding space. The vector representation 226 includes multiple features in the learned embedding space. In some embodiments, generating the vector representation 226 includes applying the mapping from the trained embedding model 208 to a node in the taxonomic graph 204 indicative of the respective species.

The sequence analyzer 210 is configured to determine a plurality of k-mers from one or more sequence reads. The sequence reads are indicative of a whole genome of a organism. The sequence reads may be included in sequence data 222, which may be received from the sequencer 104, the bioinformatics platform 106, and/or another source of sequence data 222. Each of the sequence reads may include a nucleotide sequence such as a DNA sequence, a 16S RNA sequence, a 23S RNA sequence, or a metagenome sequence. Each k-mer includes an atomic sub-sequence of nucleotide bases having a predetermined length. In some embodiments, determining the k-mers includes selecting one or more k-mers from each sequence read using a sliding window of the predetermined length and a predetermined stride. The sequence analyzer 210 is further configured to determine a relative co-occurrence between each pair of k-mers of the plurality of k-mers. In some embodiments, determining the relative co-occurrence includes iteratively updating the relative co-occurrence for each pair of k-mers with an update function. In the update function, an initial relative co-occurrence value for each pair of k-mers is set to zero, the relative co-occurrence value for each pair of k-mers is set to one when the pair of k-mers is first observed, and the relative co-occurrence value for each pair of k-mers is increased when the pair of k-mers is subsequently observed. Output of the update function is bound by a weighted update function.

The pseudo-image factory 212 is configured to construct a pseudo-image 224 as a function of the relative co-occurrence between each pair of k-mers. Each pixel of the pseudo-image 224 has a value indicative of the relative co-occurrence for an associated pair of k-mers. In some embodiments, constructing the pseudo-image 224 includes constructing multiple intermediate pseudo-images for each corresponding sequence read and summing pixel values of the intermediate pseudo-images to generate the pseudo-image 224. In some embodiments, constructing the pseudo-image 224 includes concatenating the sequence reads to generate a concatenated sequence read and constructing the pseudo-image 224 with the concatenated sequence read. In some embodiments, constructing the pseudo-image 224 includes constructing multiple intermediate pseudo-images for each corresponding sequence read, constructing another group of intermediate pseudo-images that are each for the reverse complement of a corresponding sequence read, and summing pixel values of both groups of intermediate pseudo-images to generate the pseudo-image 224.

The classifier model trainer 214 is configured to train a classifier model 218 to map a pseudo-image 224 indicative of a whole genome of an organism to a corresponding vector representation 226 in the embedding space. The classifier model 218 may be embodied as, for example, a convolutional neural network. The classifier 216 is configured to determine a vector representation 226 of species associated with a whole genome by inputting a pseudo-image 224 representing the whole genome to the classifier model 218 in response to training the classifier model 218.

The classification analyzer 220 is configured to search for a node in the hierarchical taxonomy based on a distance in the embedding space between the vector representation 226 returned by the classifier model 218 and another vector representation 226 of another node. For example, each of the nodes may represent different species.

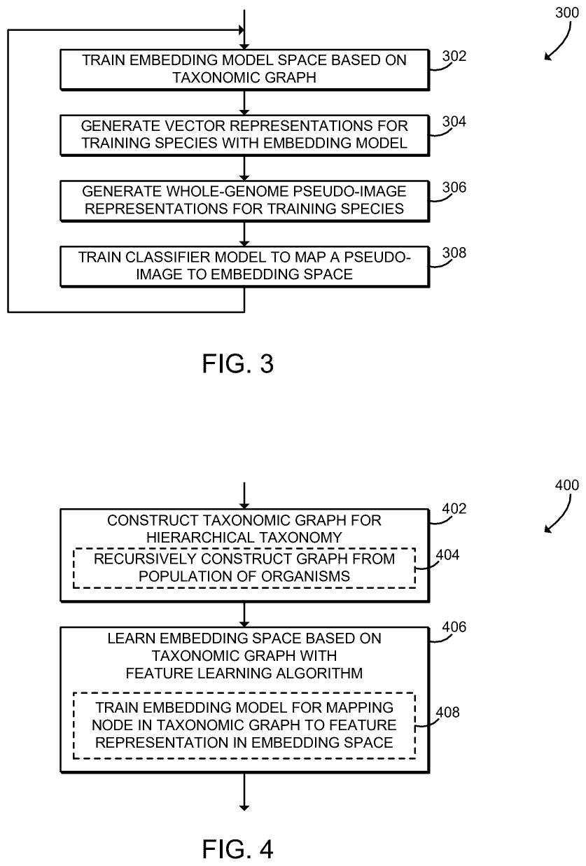

Referring now to FIG. 3, in use, the computing device 102 may execute a method 300 for embedding and classification model training. It should be appreciated that, in some embodiments, the operations of the method 300 may be performed by one or more components of the environment 200 of the computing device 102 as shown in FIG. 2. The method 300 begins with block 302, in which the computing device 102 trains the embedding model 208 based on a taxonomic graph 204. The taxonomic graph 204 may be constructed from a relatively large population of species, based on the phylogenetic or other taxonomic relationships between those species. One potential embodiment of a method for training the embedding model 208 is described below in connection with FIG. 4.

In block 304, the computing device 102 generates vector representations 226 of training species using the trained embedding model 208. These vector representations 226, or embeddings, provide taxonomy-aware, hierarchy-based representations for each species. The species used in the training data set may be sparse—that is, there may be fewer species included in the training data set as compared to the total species identified in the taxonomic graph 204.

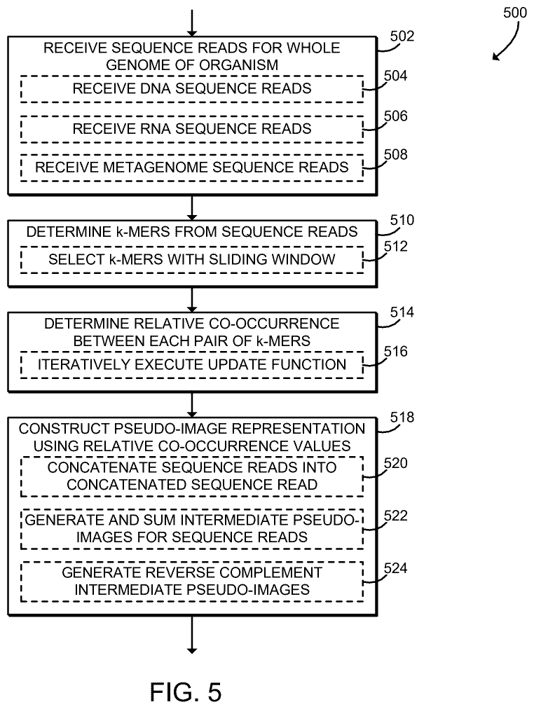

In block 306, the computing device 102 generates a whole-genome pseudo-image 224 representation for each of the training species. The pseudo-image 224 representation captures inter- and intra-species variations at the nucleotide read level. As described further below, the computing device 102 captures the relative co-occurrence between k-mers, or smaller sub-sequences of each genome, in a histogram-based representation and create a pseudo-image based on the representation. The intensity of each pixel in the pseudo-image 224 represents the relative frequency with which two k-mers (or nucleotide subsequences) co-occur in a genome, with the resulting pseudo-image forming a species-specific pattern that captures the taxonomic similarity between species. One potential embodiment of a method for generating pseudo-image representations is described below in connection with FIG. 5.

In block 308, the computing device 102 trains the classifier model 218 to map pseudo image 224 representations to vector representations 226 using the data for the training species. Given the two representations of the species, i.e., genome-level pseudoimages 224 to capture phylogenetic variations at the nucleotide level and the vector representation 226 in the taxonomy-aware, hierarchical embedding space, training the classification model 218 finds a mapping function φ:I( [symbol] ᵢ)→εᵢ that can map from the genome-level pseudo-image I( [symbol] ᵢ) of each species sᵢ∈ [symbol] to its corresponding taxonomic embedding εᵢ, such that the genome representations possess the same compositional properties.

The classification model 218 may be embodied as a convolutional neural network (CNN), a vision transformer, an attention-based reasoning model, or other classification model. In the illustrative embodiment, the classification model 218 is 10-layer CNN, where 5 blocks of 2 CNN layers each are interspersed with a max-pooling operation. A global average pooling layer is added at the end of the last convolutional block, followed by three fully connected layers to project down to the same dimension as the species-specific embedding. The network is trained to predict the taxonomy-aware embedding and capture the inter- and intra-class variations encoded within the taxonomy-aware embedding. The mapping function may be formulated as a regression function from the pseudo-images I( [symbol] ᵢ) to the embedding space εᵢ. Hence, the training may be an optimization for Equation 1, below, where W_{φ} is the set of learnable weights for the mapping function φ(.) (e.g., the classification model 218), and Et is a real-valued species-level d-dimension embedding (e.g., produced by the embedding model 208). In an embodiment, all hyperparameters were found using a grid search and kept constant for all experiments, and all networks were trained for 500 epochs.

$$ℒ = {min}_{{W}_{ϕ}} {‖ {W}_{ϕ} I ( {𝒢}_{i} ) - {ε}_{i} ‖}_{d}^{2} \qquad ( 1 )$$

In an embodiment, to aid in the computation, an L2 regularization layer was added to both the learned embedding εᵢ and the output of the mapping function φ(.). This formulation, while simple, has several advantages. First, the task of capturing phylogenetically relevant features is offloaded to learn the embedding space. Hence, during training, there is no need to compute expensive mining of hard negative (or positive) pairs. Second, by formulating the mapping function as regressing to the hierarchical taxonomic embedding space, the vocabulary of the unseen classes can be expanded by adding the embedding during inference. Further, since the target embedding space is computed independently and fixed when training the mapping function, there is no need for additional supervision, such as in center loss.

After training, the method 300 loops back to block 302, in which additional training may be performed. Additionally or alternatively, after performing model training, the trained embedding model 208 and/or the trained classification model 218 may be deployed for inference. One potential embodiment for a method of classifying whole genome sequences of organisms using the trained models 208, 218 is described below in connection with FIG. 7.

Referring now to FIG. 4, in use, the computing device 102 may execute a method 400 for learning a taxonomy-aware embedding space. As described above, the method 400 may be executed in connection with block 302 of the method 300 as shown in FIG. 3. It should be appreciated that, in some embodiments, the operations of the method 400 may be performed by one or more components of the environment 200 of the computing device 102 as shown in FIG. 2. The method 400 begins with block 402, in which the computing device 102 constructs the taxonomic graph 204 for the hierarchical taxonomy. The graph 204 represents the taxonomic relationships between species. Each species can be characterized by its taxonomic lineage, which represents the hierarchical description of the organism based on shared characteristics with other organisms. The taxonomy assigns organisms to different levels, including life, domain, kingdom, phylum, class, order, family, genus, species, and optionally sub-species, reflecting the phylogenetic relationships between organisms. In the illustrative embodiment, nodes in the graph 204 represent the taxonomy at different levels (e.g., species, genus, etc.) and edges in the graph 204 represent relationships between levels of the taxonomy.

In some embodiments, in block 404 the computing device 102 recursively constructs the taxonomic graph 204 from a population of organisms. For example, the computing device 102 may iteratively add nodes to the graph 204 based on the species taxonomy, starting from an empty graph 204, to capture the inherently compositional relationships between nodes.

In an illustrative embodiment, the taxonomic graph 204 was generated using a 72,378 species from the “bacteria” subset from NCBI Taxonomy database as the embedding search space to build the taxonomy graph 204 to capture the compositional, phylogenetic relationships among all bacterial species.

In block 406, the computing device 102 learns the embedding space based on the taxonomic graph 204 using a feature learning algorithm. Learning the embedding space captures the compositional properties inherent in the graph 204. In some embodiments, in block 408 the computing device 102 trains the embedding model 208 for mapping a node in the taxonomic graph 204 (e.g., representing a species) to a vector representation 226 in the feature space.

In the illustrative embodiment, the computing device 102 uses the node2vec algorithm as the feature learning algorithm. The node2vec algorithm is used to learn a low-rank embedding space. The node2vec algorithm offers a flexible objective that preserves the neighborhood structure for each node and captures it in the embedding space using simulated biased random walks. This formulation captures the phylogenetic relationships between species as described above. By incorporating hyperparameters such as the return and in-out parameters, node2vec combines depth-first search (DFS)-like and breadth-first search (BFS)-like neighborhood exploration and employs negative sampling to optimize a Skip-gram model 208.

In the illustrative embodiment, the taxonomic graph 204 including 72,378 species was used to learn the embedding space with node2vec. Ten random walks were generated for each species, with a maximum length of 100 visited nodes in each walk to generate the context. Having longer random walks allows the similarity in neighborhood structure to be captured for both highly related (such as from the same genus) and distantly related (i.e., from a different family) species. The initial learning rate was set to be 4×10⁻⁵, and a cold start was first performed, i.e., for five epochs, the learning rate is set to be 4×10⁻¹² and then increased.

After learning the embedding space, the method 400 is completed. As described above, the trained embedding model 208 may be used to generate vector representations 206 for one or more species included in a training data set, which may be used to train the classification model 218.

Referring now to FIG. 5, in use, the computing device 102 may execute a method 500 for constructing a pseudo-image representation of a whole organism genome. As described above, the method 500 may be executed in connection with block 306 of the method 300 as shown in FIG. 3. Further, the method 500 may also be executed in connection with the method of FIG. 7, described below. It should be appreciated that, in some embodiments, the operations of the method 500 may be performed by one or more components of the environment 200 of the computing device 102 as shown in FIG. 2. The method 500 begins with block 502, in which the computing device 102 receives sequence reads for a whole genome of an organism. Each whole genome ( [symbol] ᵢ) may be considered to be a collection of nucleotides drawn from a set of bases {A, T,C,G}, sequenced as “reads” (X₀, X₁, . . . , Xₙ∈ [symbol] ᵢ). The sequence reads, for example included in sequence data 222, may be received from the sequencer 104, from the bioinformatics platform 106, or from another sequence data source. In some embodiments, in block 504 the computing device 102 receives DNA sequence reads. In some embodiments, in block 506 the computing device 102 receives RNA sequence reads (e.g., 16S RNA, 23S RNA, or another RNA sequence). In some embodiments, in block 508 the computing device 102 receives metagenome sequence reads.

In block 510, the computing device 102 determines a set of k-mers from the sequence reads. Each k-mer is an atomic sub-sequence of nucleotide bases from a sequence read having a predetermined length (i.e., k bases). In some embodiments, in block 512 the computing device 102 selects the k-mers using a sliding window of length k. In some embodiments, the k-mers may be selected using the sliding window of length k with a predetermined stride length s. Each k-mer may be denoted as xⱼ, where x₀, x₁, . . . , x₁∈Xi.

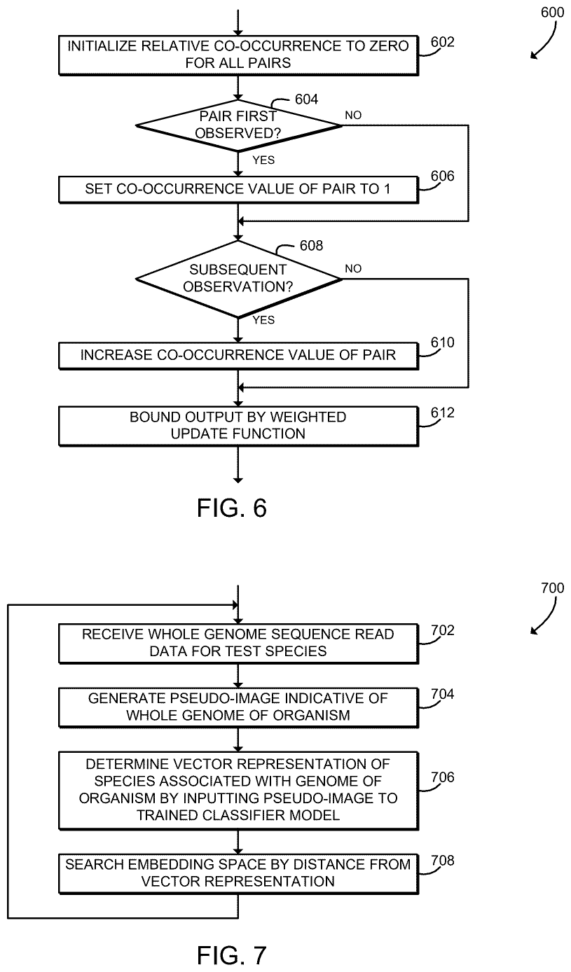

In block 514, the computing device 102 determines a relative co-occurrence value between each pair of k-mers. In some embodiments, in block 516, the computing device 102 iteratively executes an update function for each pair of k-mers in the set of generated k-mers. Formally, the update function may be defined according to Equation 2, below, where the relative co-occurrence is updated using the previously observed co-occurrence frequency e_{i,j} and the current co-occurrence frequency e′_{i,j}, between k-mers xᵢ and xⱼ. One potential embodiment of the update function is described below in connection with FIG. 6.

$${f}_{r} ( {x}_{i} , {x}_{j} ) = σ ( \frac{{e}_{i , j}}{( {‖ {e}_{i , j} - {e}_{i , j}^{′} ‖}_{2} )} ) \qquad ( 2 )$$

In block 518, the computing device 102 constructs a pseudo-image representation using the relative co-occurrence values. In the pseudo-image representation, the intensity value of each pixel ‘represents the co-occurrence value for a pair of k-mers. The pair of k-mers may be identified by the coordinates (e.g., x- and y-coordinate) of the pixel within the image. In some embodiments, pixel values may be normalized or otherwise processed, and the size of the image may be scaled or otherwise modified. In the illustrative embodiment, the pseudo-image representations are embodied as grayscale images, where each color channel is equal and representative of the relative co-occurrence values. Additionally or alternatively, in some embodiments, the pseudo-image representation may be a false color image. As yet another additional or alternative embodiment, the pseudo-image representation may be a hyperspectral image, for example including additional channels that represent other quantities, such as co-occurrence values for k-mers of differing length k. In an illustrative embodiment, the k-mer length k was set to 6 with a stride s of 10, and the pseudo-images were resized to 512×512 pixels.

In an illustrative embodiment, the pseudo-image Iᵣ is constructed by considering each pixel p_{i,j}∈Ir to represent the relative co-occurrence of k-mers xᵢ and xⱼ. This formulation allows the whole genome to be represented as an image and thus enables capture of long-range properties that have proven to be hard for sequence-based learning models such as LSTMs and Transformers. Additionally, the pseudo-image representation may leverage advances in computer vision, such as convolutional neural networks (CNNs), that can effectively capture the spatial relationships for effective representation learning.

The resulting pseudo-image representation is given by Equation 3, below, where the pseudo-image is constructed by iterating over all possible k-mer combinations for a given window length k and stride s. Each pixel Iᵣ(i,j) is assigned the output of fᵣ(xᵢ, xⱼ) from Equation 2, above. The co-occurrences are normalized to be between 0 and 1 using N, which is the sum of all k-mer co-occurrences from all sequences in the genome. A cutoff parameter, λₘ, is introduced to remove any spurious patterns that can be introduced due to sequencing errors, which can flip base pairs randomly. An RGB image may be simulated to leverage standard CNN architectures for image processing by duplicating each pixel value to create an image depth of 3. In the illustrative embodiment, the cutoff parameter λₘ was set to zero.

$${I}_{r} ( i , j ) = {\sum }_{i = 1}^{{4}^{k}} {\sum }_{j = i + s}^{{4}^{k}} \{ \begin{matrix}255 * \frac{{f}_{r} ( i , j )}{N} , if {f}_{r} ( {x}_{i} , {x}_{j} ) > {λ}_{m} \\ 0 , otherwise\end{matrix} \qquad ( 3 )$$

The illustrative computing device 102 may employ one or more different strategies for constructing the pseudo-image representation 224. In some embodiments, in block 520 the computing device 102 concatenates the sequence reads for the whole genome into a concatenated sequence read, and then generates the pseudo-image representation based on the concatenated sequence read. In some embodiments, in block 522 the computing device 102 generates an intermediate pseudo-image for each individual sequence read, and then sums the pixel values of the intermediate pseudo-images to generate the pseudo-image representation 224. The resulting image is normalized by the sum of all genome-level k-mer co-occurrences, scaling each pixel value between 0 and 1. In some embodiments, in block 524, the computing device 102 generates intermediate pseudo-image representations for the reverse complement of each sequence read. The intermediate pseudo-image representations for the reverse complements may be summed with other intermediate pseudo-image representations of the sequence reads to generate the pseudo-image representation 224, thus capturing the symmetry inherent in DNA structures. This strategy allows for additional cues in capturing phylogenetic variations at the genome level.

After constructing the pseudo-image representation 224, the method 500 is completed. The method 500 may be executed again for additional whole genomes. The constructed pseudo-image representations 224 may be used for model training as described above in connection with the method of FIG. 3 and/or may be used for model inference as described below in connection with the method of FIG. 7.

Referring now to FIG. 6, in use, the computing device 102 may execute a method 600 for iteratively executing an update method. As described above, the method 600 may be executed in connection with block 514 of the method 500 as shown in FIG. 5. It should be appreciated that, in some embodiments, the operations of the method 600 may be performed by one or more components of the environment 200 of the computing device 102 as shown in FIG. 2. The method 600 begins with block 602, in which the computing device 102 initializes the relative co-occurrence value to zero for all pairs of k-mers. Accordingly, if a pair of k-mers never co-occur, then the frequency remains at zero.

In block 604, the computing device 102 evaluates a pair of k-mers passed to the update function and determines whether that pair is being observed for the first time. For example, the computing device 102 may determine whether the co-occurrence value for that pair equals the initial value (i.e., zero). If the pair is not being observed for the first time, the method 600 branches ahead to block 608. If the pair is being observed for the first time, the method 600 advances to block 606, in which the co-occurrence value of the pair is set to one.

In block 608, the computing device 102 determines whether the current pair of k-mers has been previously observed (i.e., this is a subsequent observation). For example, the computing device 102 may determine whether the co-occurrence value for that pair does not equal the initial value (i.e., nonzero). If not (e.g., if the pair is being observed for the first time), the method 600 branches ahead to block 612. If the pair is being subsequently observed, the method 600 advances to block 610, in which the computing device 102 increases the co-occurrence value of the pair.

In block 612, the computing device 102 bounds the output of the update function by a weighted update function. For example, the computing device 102 may bound the co-occurrence value to between −2 and +2. In an illustrative embodiment, a weighted update function σ(.) was set as shown in Equation 4, below. Additionally, this formulation allows spurious patterns that could be a product of noise to be suppressed while highlighting structures in the genome that are produced due to frequent co-occurrences of k-mers within the entire genome sequence. After bounding the output, the method 600 is completed. As described above, the method 600 may be iteratively executed for each pairing of k-mers in a sequence read. Output values after iteration is complete are used to generate the pseudo-image representation 224.

$$σ ( r ) = 2 \sqrt{max ( r - 1 , 1 )} + ( min ( r - 2 , 2 ) + 2 ) \qquad ( 4 )$$

Referring now to FIG. 7, in use, the computing device 102 may execute a method 700 for genome classification using pseudo-image representations and a taxonomy-aware embedding space. It should be appreciated that, in some embodiments, the operations of the method 700 may be performed by one or more components of the environment 200 of the computing device 102 as shown in FIG. 2. The method 700 begins with block 702, in which the computing device 102 receives whole genome sequence read data 222 for a test or query species. In a zero-shot learning embodiment, the test species was not included in the training data set used to train the classifier model 218 as described above.

In block 704, the computing device 102 generates a pseudo-image representation 224 indicative of the whole genome of the organism using the sequence read data 222. The computing device 102 may, for example, generate the pseudo-image representation 224 using the method 500 shown in FIG. 5 and described above.

In block 706, the computing device 102 determines a vector representation 216 for the test species associated with the whole genome by inputting the pseudo-image representation 224 to the classification model 218. The classification model 218 is trained, for example using the method 300 of shown in FIG. 3 and described above. The classification model 218 returns a vector representation 226, which may be embodied as a collection of coordinates in the taxonomy-aware embedding space described above.

In block 708, the computing device 102 searches the embedding space by distance from the vector representation 226 returned from the classifier model 218. As described above, distance in the embedding space is indicative of similarity between genomes. Additionally, the embedding space also indicates the taxonomic lineage for the query genome. Thus, in a zero-shot learning scenario, the vector representation 226 may be used to find related species, genus, or other taxonomic levels related to a previously unseen query genome, not included in the training data. After searching the embedding space and/or otherwise analyzing the vector representation 226, the method 700 loops back to block 702, in which the computing device 102 may classify further whole genome sequence reads.

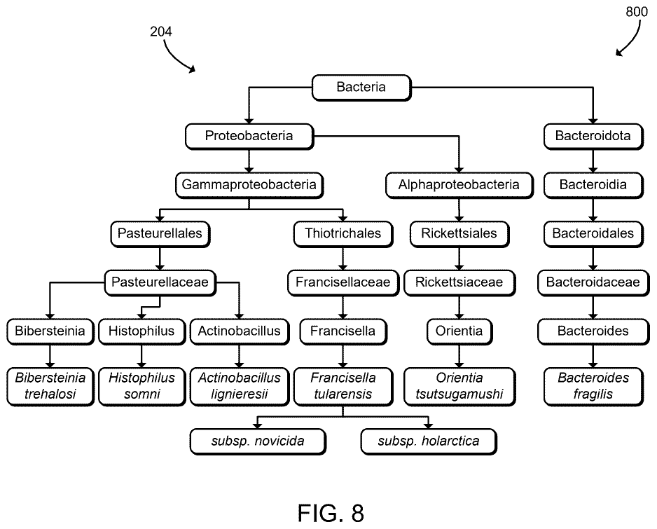

Referring now to FIG. 8, diagram 800 illustrates one potential embodiment of a taxonomic graph 204 that may be generated by the computing device 102. The illustrative graph 204 may be recursively generated from in a top-down manner, starting from the kingdom (or domain) level and progressing to the species level, and optionally to the sub-species level if applicable. For example, starting from an empty graph, given a species such as Bibersteinia trehalosi, the computing device 102 may populate the graph 204 with nodes for the kingdom bacteria, the phylum proteobacteria, and so on, to the species Bibersteinia trehalosi. The computing device 102 may then iteratively add nodes to the graph 204 for the remaining species to arrive at the graph shown in the diagram 800.

As shown, traversing the graph 204 from one species node to another using a random walk reveals the phylogenetic similarity between the two species. The length of the walk reflects their similarity, while the path taken provides information on their relationship. For instance, in FIG. 8, we can infer that the representations for Bibersteinia trehalosi and Actinobacillus lignieresii should be closer together than to Francisella tularensis since the former two belong to the same family (Pasteurellaceae) and can be reached within three steps. However, all three species belong to the same class Gammaproteobacteria, and hence, they can be reached within eight steps.

In an experiment, a query genome for Francisella tularensis subsp. novicida was classified as described above. The query genome was unseen, that is, not included in training data for the classification model 218. Although the subspecies novicida was unseen, the disclosed approach was able to retrieve very relevant neighbors (the top 3 predictions were Francisella tularensis subsp. novicida, Francisella tularensis, and Francisella tularensis subsp. holarctica) while placing the correct subspecies at the top-1 prediction. This performance is remarkable considering that the other subspecies holarctica was part of the training set, yet was not the top prediction. This result indicates that subtle variations at both genome and taxonomy levels are captured by the system 100. In another experiment, the system 100 was able to retrieve highly relevant species to a genome query from an unseen genus (Biberstinia). Interestingly, in that experiment all top-5 predictions were placed within the same family Pasteurellaceae of the target species, with the correct prediction at the third position on the prediction order. Thus, the proposed embedding successfully guides taxonomy navigation and provides highly relevant predictions for a genome whose corresponding genus (e.g., Bibersteinia) and species (e.g., Bibersteinia trehalosi) were not observed during training.

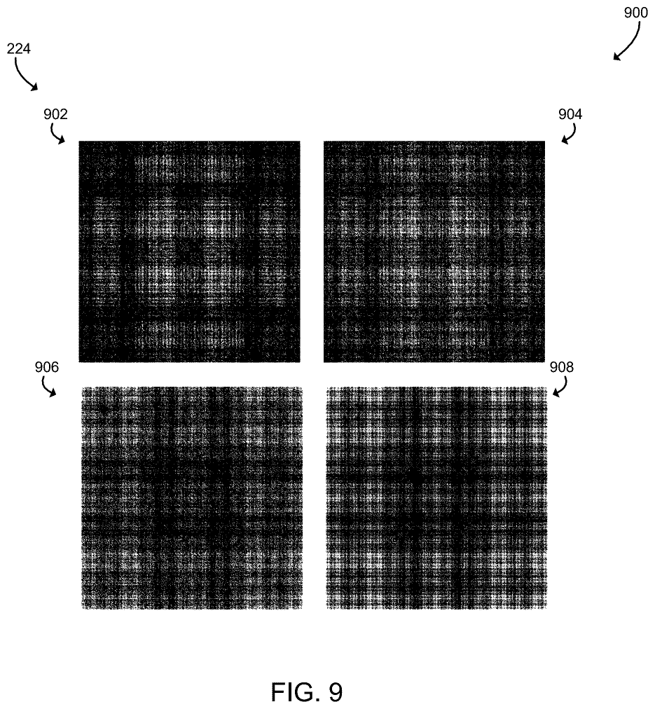

Referring now to FIG. 9, diagram 900 illustrates potential embodiments of pseudo-image representations 224 that may be generated by the computing device 102. In an illustrative embodiment, the pseudo-images 224 of the diagram 900 were generated for whole genomes of bacterial organisms, considering both sequence reads and reverse complements of the sequence reads, as described above in connection with blocks 522, 524 of FIG. 5. Pseudo-image 902 represents Mycobacterium avium, pseudo-image 904 represents Mycobacterium tuberculosis, pseudo-image 906 represents Francisella tularensis, and pseudo-image 908 represents Clostridium botulinum. Note that pseudo-images 902, 904, representing species from the same genus Mycobacterium, include similar patterns that are readily apparent.

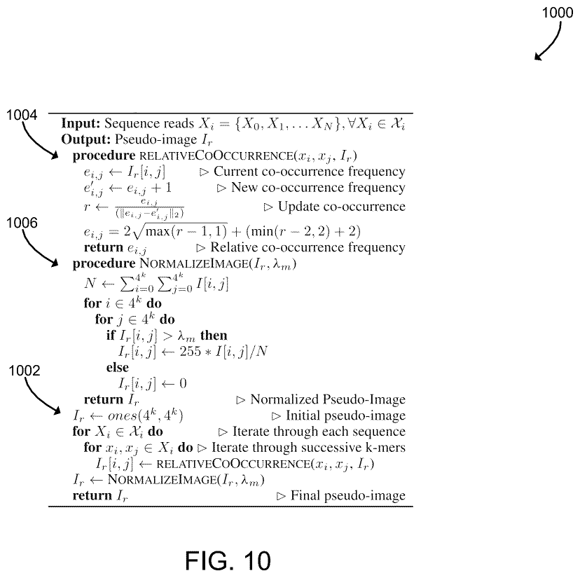

Referring now to FIG. 10, pseudocode diagram 1000 illustrates one possible embodiment of a pseudo-image 224 construction process. As shown, the process 1000 starts at statement 1002, in the pseudo-image is initialed. Next, the process 1000 iterates over each sequence read in the genome, and then further iterates through successive pairs of k-mers in each sequence read. The pixel value for each pair of k-mers is updated with the update function, which is illustratively a procedure 1004, labeled as RelativeCoOccurence. As shown, the update function 1004 increases the co-occurrence value each time a pair of k-mers is recognized, with the output bound between −2 and +2. After iterating through all pairs of k-mers for all sequence reads, the process 1000 next normalizes the pseudo-image using a procedure 1006, labeled as NormalizeImage. The normalize image procedure 1006 normalizes each image pixel between 0 and 1 using N, which is the sum of all k-mer co-occurrences from all sequences, and applies a cutoff parameter λₘ. The procedure 1000 returns the normalized pseudo-image I.

1. A computing device for representing a genomic sequence, the computing device comprising:
a sequence analyzer to (i) determine a plurality of k-mers from a plurality of sequence reads, wherein the plurality of sequence reads is indicative of a first whole genome of a first organism, and wherein each k-mer comprises an atomic sub-sequence of nucleotide bases having a predetermined length, and (ii) determine a relative co-occurrence between each pair of k-mers of the plurality of k-mers; and
a pseuodo-image factory to construct a first pseudo-image as a function of the relative co-occurrence between each pair of k-mers, wherein each pixel of the first pseudo-image has a value indicative of the relative co-occurrence for an associated pair of k-mers.

2. The computing device of claim 1, wherein to construct the first pseudo-image comprises to:
construct a plurality of intermediate pseudo-images, wherein each intermediate pseudo-image is generated for a corresponding sequence read; and
sum pixel values of the plurality of intermediate pseudo-images to generate the first pseudo-image.

3. The computing device of claim 1, wherein to construct the first pseudo-image comprises to:
concatenate the plurality of sequence reads to generate a concatenated sequence read; and
construct the first pseudo-image with the concatenated sequence read.

4. The computing device of claim 1, wherein to construct the first pseudo-image comprises to:
construct a plurality of intermediate pseudo-images, wherein each intermediate pseudo-image of the plurality of intermediate pseudo-images is generated for a corresponding sequence read;
construct a second plurality of intermediate pseudo-images, wherein each intermediate pseudo-image of the second plurality is generated for a reverse complement of a corresponding sequence read; and
sum pixel values of the plurality of intermediate pseudo-images and the second plurality of intermediate pseudo-images to generate the first pseudo-image.

5. The computing device of claim 1, wherein to determine the plurality of k-mers comprises to select one or more k-mers from each sequence read using a sliding window of the predetermined length and a predetermined stride.

6. The computing device of claim 1, wherein to determine the relative co-occurrence between each pair of k-mers comprises to iteratively update the relative co-occurrence for each pair of k-mers with an update function.

7. The computing device of 6, wherein in the update function, an initial relative co-occurrence value for each pair of k-mers is set to zero, wherein the relative co-occurrence value for each pair of k-mers is set to one when the pair of k-mers is first observed, wherein the relative co-occurrence value for each pair of k-mers is increased when the pair of k-mers is subsequently observed, and wherein output of the update function is bound by a weighted update function.

8. The computing device of claim 1, wherein each of the sequence reads comprises a DNA sequence.

9. The computing device of claim 1, wherein each of the sequence reads comprises a 16S RNA sequence, a 23S RNA sequence, or a metagenome sequence.

10. A computing device for classification of organisms by species, the computing device comprising:
an embedding model trainer to learn an embedding space based on a taxonomic graph with a feature learning algorithm, wherein the taxonomic graph comprises a plurality of nodes indicative of levels in a hierarchical taxonomy and a plurality of edges that connect nodes;
a classifier model trainer to train a classifier model to map a pseudo-image indicative of a whole genome of an organism to a corresponding vector representation in the embedding space;
a pseudo-image factory to generate a first pseudo-image indicative of a first whole genome of a first organism; and
a classifier to determine a first vector representation of a first species associated with the first whole genome by input of the first pseudo-image to the classifier model in response to training of the classifier model.

11. The computing device of claim 10, wherein
wherein to learn the embedding space comprises to learn a mapping from a node in the taxonomic graph to a feature representation in the embedding space; and
the computing device further comprises an embedding mapper to generate the corresponding vector representation of the whole genome of the organism in response to learning of the embedding space, wherein the corresponding vector representation comprises a plurality of features in the embedding space, and wherein to generate the corresponding vector representation comprises to apply the mapping to a first node in the taxonomic graph indicative of the whole genome of the organism.

12. The computing device of claim 10, wherein the hierarchical taxonomy comprises a taxonomy of organisms reflecting phylogenetic relationships between the organisms.

13. The computing device of claim 10, wherein the embedding model trainer is further configured to construct the taxonomic graph, wherein to construct the taxonomic graph comprises to recursively construct the taxonomic graph based on a predetermined plurality of organisms.

14. The computing device of claim 10, wherein the classifier model comprises a convolutional neural network.

15. The computing device of claim 10, further comprising a classification analyzer to search for a second node in the hierarchical taxonomy based on a distance in the embedding space between the first vector representation and a second vector representation of the second node.

16. The computing device of claim 15, wherein the second node comprises a second species.

17. A method for representing a genomic sequence, the method comprising:
determining, by a computing device, a plurality of k-mers from a plurality of sequence reads, wherein the plurality of sequence reads is indicative of a first whole genome of a first organism, and wherein each k-mer comprises an atomic sub-sequence of nucleotide bases having a predetermined length;
determining, by the computing device, a relative co-occurrence between each pair of k-mers of the plurality of k-mers; and
constructing, by the computing device, a first pseudo-image as a function of the relative co-occurrence between each pair of k-mers, wherein each pixel of the first pseudo-image has a value indicative of the relative co-occurrence for an associated pair of k-mers.

18. The method of claim 17, wherein constructing the first pseudo-image comprises:
concatenating the plurality of sequence reads to generate a concatenated sequence read; and
constructing the first pseudo-image with the concatenated sequence read.

19. The method of claim 17, wherein constructing the first pseudo-image comprises:
constructing a plurality of intermediate pseudo-images, wherein each intermediate pseudo-image of the plurality of intermediate pseudo-images is generated for a corresponding sequence read;
constructing a second plurality of intermediate pseudo-images, wherein each intermediate pseudo-image of the second plurality is generated for a reverse complement of a corresponding sequence read; and
summing pixel values of the plurality of intermediate pseudo-images and the second plurality of intermediate pseudo-images to generate the first pseudo-image.

20. The method of claim 17, wherein determining the relative co-occurrence between each pair of k-mers comprises iteratively updating the relative co-occurrence for each pair of k-mers using an update function.
